(19) United States
(12) Patent Application Publication
(10) Pub. No.: US 2026/0297142 A1
(43) Pub. Date: Oct. 1, 2026

(54) PHOTOCROSSLINKABLE RECOMBINANT BACTERIAL COLLAGEN-LIKE PROTEINS

(71) Applicant: Evonik Operations GmbH, Essen (DE)

(72) Inventors: Yunxiao LIU, Singapore (SG); Eva Yihua LOO, Singapore (SG); Ann RITTER, Mülheim an der Ruhr (DE)

(73) Assignee: Evonik Operations GmbH, Essen (DE)

(21) Appl. No.: 18/880,442

(22) PCT Filed: Jun. 21, 2023
(86) PCT No.: PCT/EP2023/066741

§ 371 Date: Dec. 31, 2024

(30) Foreign Application Priority Data
Jul. 1, 2022 (EP) 22182499.8
Feb. 8, 2023 (EP) 23155606.9

(51) Int. Cl. C07K 14/315; A61L 27/20; A61L 27/22; A61L 27/52

(52) U.S. Cl. CPC C07K 14/315; A61L 27/20; A61L 27/227; A61L 27/52

(57) ABSTRACT

Recombinant bacterial collagen-like protein with an amino acid sequence that is at least ≥60% identical to the amino acid sequence of SEQ ID NO:1 characterized in that the amino acid sequence comprises a deletion of at least 38 amino acids at the N-terminus of the amino acid sequence of SEQ ID NO:1 and that the recombinant collagen-like protein is functionalized with ethylenic unsaturated groups.

The invention pertains to photocrosslinkable functionalized recombinant collagen-like proteins (CLPs) of bacterial origin which can be applied as a bioink for cell encapsulation, as well as preparation of 3D scaffolds, in vitro models and tissue grafts.

Bioinks which are commonly used for bioprinting, are largely synthetic thermoplastic polymers or animal-derived proteins e.g. collagen, gelatin, (meth)acryloyl functionalized animal derived collagen or -gelatin methacrylate.

US 2016/0051727 discloses a collagen based polymeric material comprising collagen molecules and/or collagen derived molecules which are functionalized by the addition of one or more ethylenically unsaturated groups and which are cross-linked via said groups.

US 2020/0179562 discloses curable recombinant human collagen functionalized with methacryloyl which can be formulated into inks for additive manufacturing and printed into 3 dimensional objects.

US 2016/0193384 discloses the production of a hydrogel by photocrosslinking a methacryloyl or acryloyl collagen and a synthetic polymer. Such hydrogels can be used as 3D scaffolds and implants, suitable as inks for 3D printing in order to prepare complex 3D structures, or for incorporating cells into the structure.

The collagen in the above referenced prior art is obtained from human, mammalian or avian sources. Such human or animal-derived collagens are not well defined, viscous and have poor aqueous solubility under physiological conditions. Due to the viscosity, significant force is needed to extrude or jet the viscous collagen mixture during the printing process. This results in greater shear stress (and thus lower cell viability) if cells are co-printed in the same mixture. The low pH needed to solubilize animal-derived collagen also makes it challenging to increase the concentration.

It is therefore an object of the present invention to provide a protein that can be formulated into a bioink and crosslinked to a hydrogel and which can at least reduce the disadvantages of prior art materials in particular animal derived collagens.

This object is achieved by recombinant bacterial collagen-like proteins with an amino acid sequence that is at least ≥60% identical to the amino acid sequence of SEQ ID NO:1, characterized in that the amino acid sequence comprises a deletion of at least 38 amino acids at the N-terminus of the amino acid sequence of SEQ ID NO:1 and that the recombinant collagen-like protein is functionalized with ethylenic unsaturated groups.

In a bioink composition the ethylenic unsaturated groups can form polymeric crosslink networks with functionalized recombinant bacterial collagen-like protein molecules or with other components in the bioink composition in the presence of radicals, anions, nucleophiles or combinations thereof.

There is an unmet need for GMP compliant bioinks to facilitate the translation of bioprinted clinical products from bench-to-bedside, particularly water-soluble biomaterials which can form hydrogels and thus support cell encapsulation and bioprinting.

Collagen-like proteins (CLPs) of bacterial origin have interesting mechanical properties, similar to those of higher eukaryotes' collagen proteins, without needing the complex maturing steps required for the eukaryotic counterparts. CLPs present a common structure: two alpha helixes, stabilizing each other, constitute a “V domain”, which is followed by a rod-like, structural collagen domain (CL). After the collagen domain, typically a membrane anchor (GPI-like) is present at the C-terminal end of the protein.

The most industrially relevant CLP being the product of Streptococcus pyogenes and designated Scl2.

As described in various publications (Lukomski et al. 2002, Brodsky et al. 2009) the current understanding of this process is that the V-domain is required for folding three Scl2 protein monomers into one triple helical structure in vitro.

Since the V-domain was thought to be crucial for production of triple helical Scl2 it was never considered to remove this sequence leading to the following challenges:

The V-domain makes up for approximately one third of the whole sequence and hinders the protein to be transported out of the Pichia pastoris host. This requires a complex downstream process containing cell lysis to remove the target protein from the cell.

The V-domain itself has pathogenic properties and needs to be removed during the purification process. This is done by a protease digest. Usage of a protease is quite costly, and it needs to be removed during downstream as well.

It was a surprising finding that truncated variants of the collagen-like protein, including variants with a truncated V-domain or without any V-domain lead to increased production of collagen-like protein and secretion into the fermentation medium. It was further surprising that the truncated variants could be correctly folded in absence of the V-domain.

It is preferred, when the amino acid sequence comprises a deletion of between 38 and 74 amino acids at the N-terminus of the amino acid sequence of SEQ ID NO:1. This includes a complete deletion of the N-terminal V-domain (comprising 74 amino acids) and different truncations of the V-domain of at least 38 amino acids.

The amino acid sequence is preferably at least 60%, identical to the amino acid sequence of SEQ ID NO:2, SEQ ID NO:3 or SEQ ID NO:4. SEQ ID NO:2 and SEQ ID NO:3 are truncated versions of Scl2 and in SEQ ID NO:4 the N-terminal V-domain is completely deleted.

The sequence ID's indicated herein pertain to the amino acid sequence as such before functionalization with an ethylenically unsaturated group and not the functionalized recombinant bacterial collagen-like protein.

In a preferred configuration the amino acid sequence is at least ≥90%, ≥92%, ≥94%, ≥96%, ≥97%, ≥98%, ≥99% or 100%, preferably ≥97%, particularly preferably ≥98%, very particularly preferably ≥99%, and extremely preferably 100%, identical to the amino acid sequence of SEQ ID NO:2, SEQ ID NO:3 or SEQ ID NO:4.

In a preferred embodiment truncated variants of the collagen-like protein from Streptococcus pyogenes are used for recombinant bacterial collagen-like protein functionalized with ethylenic unsaturated groups according to the invention.

The inventive functionalized recombinant bacterial collagen-like protein correspondingly also encompasses polypeptide variants of SEQ ID NO:1 to 4, which contain one or more insertion(s) or deletion(s). Preferably, the polypeptide contains a maximum of 5, a maximum of 4, a maximum of 3, or a maximum of 2, insertions or deletions of amino acids.

Thus, the following functionalized recombinant bacterial collagen-like proteins also form part of the invention:

Recombinant bacterial collagen-like protein with an amino acid sequence at least 94% identical to the amino acid sequence of SEQ ID NO:2, SEQ ID NO:3 or SEQ ID NO:4 and the recombinant collagen-like protein is functionalized with methacrylate, acrylate, methacrylamide, acrylamide groups or mixtures thereof.

Recombinant bacterial collagen-like protein with an amino acid sequence at least 96% identical to the amino acid sequence of SEQ ID NO:2, SEQ ID NO:3 or SEQ ID NO:4 and the recombinant collagen-like protein is functionalized with methacrylate, acrylate, methacrylamide, acrylamide groups or mixtures thereof.

Recombinant bacterial collagen-like protein with an amino acid sequence at least 98% identical to the amino acid sequence of SEQ ID NO:2, SEQ ID NO:3 or SEQ ID NO:4 and the recombinant collagen-like protein is functionalized with methacrylate, acrylate, methacrylamide, acrylamide groups or mixtures thereof.

Recombinant bacterial collagen-like protein with an amino acid sequence at least 99% identical to the amino acid sequence of SEQ ID NO:2, SEQ ID NO:3 or SEQ ID NO:4 and the recombinant collagen-like protein is functionalized with methacrylate, acrylate, methacrylamide, acrylamide groups or mixtures thereof.

Recombinant bacterial collagen-like protein according to amino acid sequences SEQ ID NO:2, SEQ ID NO:3 or SEQ ID NO:4, the amino acid sequence containing one to five, one to four, or one to three insertion(s) or deletion(s) and the recombinant collagen-like protein is functionalized with methacrylate, acrylate, methacrylamide, acrylamide groups or mixtures thereof.

The recombinant bacterial collagen-like proteins (CLP) can be produced in a process comprising the following steps:
a) fermentation of a bacterial, yeast or plant host cell, expressing a CLP with an amino acid sequence that is at least ≥60% identical to the amino acid sequence of SEQ ID NO:1, in a medium, wherein the amino acid sequence comprises a deletion of at least 38 amino acids at the N-terminus of the amino acid sequence of SEQ ID NO:1,
b) accumulation of the bacterial CLP in the medium, wherein a fermentation broth is obtained,
c) separating the bacterial, yeast or plant host cells from the fermentation broth,
d) incubating the fermentation broth for at least 1 h at not more than 25° C. for folding of the CLP,
e) optionally purification of the bacterial CLP by at least one of the following: solvent precipitation, tangential flow filtration (TFF), ion exchange chromatography, reversed-phase chromatography.

The invention correspondingly also relates to polypeptide variants of SEQ ID NO:1 to 4, which contain one or more insertion(s) or deletion(s). Preferably, the polypeptide contains a maximum of 5, a maximum of 4, a maximum of 3, or a maximum of 2, insertions or deletions of amino acids.

In a preferred embodiment the folding of CLP in step d) is performed at a temperature between −80° C. and 25° C., preferably between 0° C. and 20° C. In a preferred configuration folding is performed in presence of glycerin or salts.

In another preferred embodiment, folding of CLP in step d) is performed for a time between 1 h and 48 h, preferably between 1 h and 24 h.

In another preferred embodiment, folding of CLP in step d) is performed with a concentration of CLP of at least 1 mg/ml, preferably at least 4 mg/ml.

In a preferred embodiment, the host cell is a microorganism of the species P. pastoris, E. coli, P. putida or C. glutamicum comprising any of the polypeptides according to the present invention.

In a preferred embodiment, the microorganism is a yeast of the genus P. pastoris or a bacterial cell, preferably E. coli, Corynebacterium or Brevibactetium.

The microorganism may be a microorganism in which the nucleotide sequence encoding the CLP is present in overexpressed form.

### Functionalization

The recombinant bacterial collagen-like protein is functionalized with ethylenic unsaturated groups. The term “ethylenic unsaturated group” refers to formate, vinyl, allyl, itaconate, fumarate or (meth)acryloyl groups. Preferably the ethylenic unsaturated group is selected from (meth)acryloyl, fumarate or vinyl.

More preferably the recombinant bacterial collagen-like protein is functionalized with (meth)acryloyl groups. The term “(meth)acryloyl group” means either a methacrylate group, an acrylate group, a methacrylamide or an acrylamide or mixtures thereof.

If a (meth)acryloyl group is chosen for the functionalization, the functionalized recombinant bacterial collagen-like protein according to the invention can be prepared by reacting recombinant bacterial collagen-like protein that is at least ≥60% identical to the amino acid sequence of SEQ ID NO:1 and that comprises a deletion of at least 38 amino acids at the N-terminus of the amino acid sequence of SEQ ID NO:1 with (meth)acrylic anhydride in an alkaline carbonate buffer. Amino acids containing primary amine and hydroxyl groups can react with (meth)acrylic anhydride to form (meth)acrylamide and methacrylate groups respectively. The (meth)acryloyl functional group is coupled to the primary amine groups on the N-terminus, the primary amine groups of the lysine residues and the hydroxyl groups of tyrosine residues.

The degree of functionalization can be controlled via the molar ratio of the recombinant bacterial collagen-like protein and (meth)acrylic anhydride. Following the functionalization, the mixture preferably undergoes diafiltration or dialysis to remove the side products. The degree of functionalization can be determined by trinitrobenzene sulfonic acid assay, NMR, or HPLC-MS.

Due to the high solubility of the recombinant bacterial collagen-like protein in neutral and alkaline buffers, an unexpectedly high degree of functionalization exceeding 95% can be achieved with minimal or no compromise to the triple helical protein structure.

In a preferred embodiment of the invention the recombinant bacterial collagen-like protein functionalized with ethylenic unsaturated groups has a degree of functionalization ranging from 5% to 99%, preferably 50% to 99% and more preferably 60% to 99% of the primary amine groups on the N-terminus, the primary amine groups of the lysine residues and the hydroxyl groups of the tyrosine residues of the recombinant collagen-like protein.

Moreover, the degree of functionalization can be tuned by modulating the reaction conditions. By tuning the molar ratio of (meth)acrylic anhydride to recombinant collagen, (meth)acryloyl recombinant collagen with different degree of functionalization could be obtained. For example, when the ratio of (meth)acrylic anhydride to amine group of recombinant collagen was kept at 0.6:1, (meth)acryloyl recombinant collagen with a degree of functionalization around 60% was synthesized.

In comparison, animal-derived collagen is typically only 10 to 20% substituted as extreme reaction conditions would denature and hydrolyse the triple-helical protein structure, producing gelatin which is less mechanically rigid.

The invention correspondingly also relates to a process for producing a functionalized recombinant bacterial collagen-like protein using recombinant bacterial collagen-like protein with an amino acid sequence at least 98% identical to the amino acid sequence of SEQ ID NO:2, SEQ ID NO:3 or SEQ ID NO:4 and (meth)acrylic anhydride, characterized in that functionalization is carried out at a molar ratio of (meth)acrylic anhydride to amine groups of the recombinant bacterial collagen-like protein between 0.5:1 and 5:1.

The functionalization is carried out under mild conditions at room temperature and a pH of 7-10, preferably 8 to 10. Preferably the functionalization is carried out in an alkaline buffer at a buffer concentration of 0.1 M to 1 M, more preferably 0.1 M to 0.6 M, most preferably 0.1 M to 0.4 M. Preferably the alkaline buffer is a carbonate/hydrogencarbonate buffer.

The invention also relates to a process for producing a functionalized recombinant bacterial collagen-like protein using a recombinant bacterial collagen-like protein with an amino acid sequence at least 99% identical to the amino acid sequence of SEQ ID NO:2, SEQ ID NO:3 or SEQ ID NO:4 and (meth)acrylic anhydride, characterized in that functionalization is carried out at a molar ratio of (meth)acrylic anhydride to amine groups of the recombinant bacterial collagen-like protein between 0.5:1 and 5:1.

The functionalization is carried out as described above under mild conditions at room temperature and a pH of 7-10, preferably 8 to 10. Preferably the functionalization is carried out in an alkaline buffer at a buffer concentration of 0.1 M to 1 M, more preferably 0.1 M to 0.6 M, most preferably 0.1 M to 0.4 M. Preferably the alkaline buffer is a carbonate/hydrogencarbonate buffer.

The invention also relates to a process for producing a functionalized recombinant bacterial collagen-like protein using a recombinant bacterial collagen-like protein according to amino acid sequences SEQ ID NO:2, SEQ ID NO:3 or SEQ ID NO:4, the amino acid sequence containing one to five, one to four, or one to three insertion(s) or deletion(s) and (meth)acrylic anhydride, characterized in that functionalization is carried out at a molar ratio of (meth)acrylic anhydride to amine groups of the recombinant bacterial collagen-like protein between 0.5:1 and 5:1.

The functionalization is carried out as described above under mild conditions at room temperature and a pH of 7-10, preferably 8 to 10. Preferably the functionalization is carried out in alkaline buffer at a buffer concentration of 0.1 M to 1 M, more preferably 0.1 M to 0.6 M, most preferably 0.1 M to 0.4 M. Preferably the alkaline buffer is a carbonate/hydrogencarbonate buffer.

In the process of functionalization it often happens with animal or human derived collagen or collagen-like proteins disclosed in the prior art that gelatin is formed due to hydrolyzation of the collagen or collagen-like protein. This hydrolysis can happen under acidic or alkaline functionalization conditions. The gelatin has a lower molecular weight than the collagen or collagen-like protein.

Surprisingly the recombinant bacterial collagen-like protein according to the invention is not hydrolyzed into smaller sequences using the process for producing a functionalized recombinant bacterial collagen-like protein according to the invention. The combination of a recombinant bacterial collagen-like protein according to the invention and the mild functionalization conditions has the unexpected effect that the molecular weight of the recombinant bacterial collagen-like protein according to the invention does not decrease. No hydrolysis of the molecule occurs.

The thus produced functionalized recombinant bacterial collagen-like protein according to the invention is especially suitable for use in bioink compositions. Such compositions have lower viscosity compared with compositions using functionalized animal-derived collagen or prior art collagen-like proteins and therefore put less shear stress on the cells as well as reducing nozzle clogging during bioprinting. Without wishing to be bound by theory it is assumed that the unique combination of the recombinant bacterial collagen-like protein according to the invention and the process for functionalizing same partially unfolds the protein thus reducing viscosity while hydrolysis is avoided and therefore no gelatin is formed. The skilled person would have been unable to foresee this beneficial effect of the functionalization.

The functionalized recombinant bacterial collagen-like proteins according to the invention have the following advantages over animal-derived collagen with the same functionalization as exemplified by comparing recombinant bacterial collagen-like proteins functionalized with (meth)acryloyl and animal derived collagen functionalized with (meth)acryloyl groups.

| (meth)acryloyl recombinant | |
|---|---|
| bacterial collagen-like proteins | (meth)acryloyl animal |
| according to the invention | derived collagen |
| Solubility up to 100 mg/ml | Solubility up to 6 mg/ml |
| Soluble in neutral pH | Soluble only under acidic conditions |
| methacryloyl functionalization | methacryloyl functionalization is random |
| is tunable to up to 97% | and typically between 10% and 20% |
| Rigidity is tunable up to 10 kPa | Rigidity is less than 1 kPa |
| Customizable bioactivity | Intrinsic bioactivity |

The recombinant bacterial collagen-like protein (meth)acryloyl according to the invention is soluble in water, aqueous buffers at neutral or alkaline pH and in particular at physiologically relevant pH of 7 to 7.4.

Bioactivity broadly describes cell attachment and enzymatic degradation. The bioactivity can be customized via the addition of cell-interactive ligands during the photocrosslinking process.

The invention encompasses a bioink composition preferably comprising 1% to 10% weight of the recombinant bacterial collagen-like protein functionalized with ethylenic unsaturated groups. Within this concentration range, formulations containing 1.5% to 6% bioink are preferred. In the absence of additives, when dissolved in water or physiological buffers, the viscosity of the bioink composition according to the invention is below 10 Centipoise.

In addition, the bioink composition contains a photoinitiator, preferably a free radical photoinitiator. The amount of photoinitiator added to the bioink composition formulation ranges from 0.01% to 2% weight of the total liquid formulation. The photoinitiator(s) are capable of producing radicals when irradiated with actinic radiation.

The bioink composition according to the invention can be applied towards the preparation of photocrosslinked hydrogels and sponges. The invention therefore also pertains to a process for producing a hydrogel by photocrosslinking the functionalized recombinant bacterial collagen-like protein according to the invention. The bioink composition can undergo photocrosslinking to form transparent and colorless hydrogels in the presence of actinic radiation, blue or UV light. The resulting pH of the bioink composition and thus the hydrogel is preferably between 6.5 and 8. A hydrogel comprising a photocrosslinked functionalized recombinant bacterial collagen-like protein according to the invention therefore is also part of the present invention.

The bioink composition according to the invention comprises
a) 1 to 10% weight of functionalized recombinant bacterial collagen-like protein according to the invention;
b) 80 to 99% weight of aqueous solvent;
c) 0.01 to 2% weight of a photoinitiator;
d) 0 to 5% weight of photocrosslinkable polymers or other photocrosslinkable peptides;
e) 0 to 10% weight of additives, rheology modifiers, biopolymers, gelation enhancers, bioactive moieties, peptides, nanocellulose and/or cells;
   provided that the sum of all components of the bioink are 100%.

Component d) is optional as the functionalized recombinant bacterial collagen-like protein according to the invention can crosslink with itself.

Preferably the photocrosslinkable polymers of component d) are selected from natural (such as hyaluronic acid methacrylate) and synthetic (such as acrylate and methacrylate derivatives of poly(ethylene glycol)) polymers and mixed into the composition to confer additional biological properties (such as anti-fouling) and modulate the physical properties (such as degradability, and swelling behaviour in water).

Synthetic peptides with photocrosslinkable groups can be incorporated to confer additional bioactivity such as cell adhesion, stem cell differentiation and enzymatic degradability.

The bioink composition can be formulated by dissolving the functionalized recombinant bacterial collagen-like protein according to the invention in an aqueous solution, adding a water-soluble photoinitiator for example Lithium phenyl-2,4,6-trimethylbenzoylphosphinate or 1-[4-(2-Hydroxyethoxy)-phenyl]-2-hydroxy-2-methyl-1-propane-1-one.

Components d) and e) can be optionally included into the composition depending on the end application.

The bioink composition can be dispensed using a bioprinter or poured into a mould for photocuring to form hydrogel scaffolds.

Photopolymerization or photocrosslinking is preferably triggered by exposure to UV or visible light depending on the photoinitiator chosen. A process for producing a hydrogel by photocrosslinking the functionalized recombinant bacterial collagen-like protein according to the invention also forms part of the invention. Similarly, a process for producing a hydrogel by photocrosslinking the bioink formulation according the invention also forms part of the invention. Preferably the bioink formulation is photocrosslinked using UV light.

The bioink composition according to the invention has lower viscosity compared with formulations using functionalized animal-derived collagen, and therefore puts less shear stress on the cells, and less chances of the nozzle clogging during bioprinting.

Another advantage of the bioink composition according to the invention is that the mechanical properties of the resulting hydrogels are significantly more rigid compared to functionalized animal-derived collagen hydrogels and rigidity (as determined by storage moduli measured using rheology) can be adjusted by varying the concentration of the functionalized recombinant bacterial collagen-like protein according to the invention from 1% to 10% weight of the bioink composition.

This is part due to the higher solubility of the recombinant collagen, enabling the formation of high concentration hydrogels. In comparison, due to solubility considerations, bioinks derived from animal-origin collagen are typically less than 0.6%. The rigidity (as determined by storage moduli measured using rheology) of the recombinant collagen hydrogel can be as high as 8.2 kPa, as exemplified by the 9% hydrogel measured under strain conditions of 0.1%. Hydrogels comprising a photocrosslinked functionalized recombinant bacterial collagen-like protein according to the invention are therefore also part of the invention.

The additives that can be incorporated into the bioink formulation as component e) include rheology modifiers, gelation enhancers and/or bioactive moieties. These additives can enhance the mechanical, viscoelastic and biological properties of the bioink composition and/or the resulting photocrosslinked hydrogel.

Self-assembling peptides and biopolymers such as nanocellulose can also be incorporated in the bioink composition as component e). Such molecules enhance the gelation and confer additional mechanical properties. Therefore, the invention also pertains to a hydrogel comprising a photocrosslinked functionalized recombinant bacterial collagen-like protein according to the invention the hydrogel further comprising nanocellulose, peptides or mixtures thereof.

Cells can be encapsulated in the photocrosslinking process by adding them into the bioink composition. Cells can also be subsequently incorporated into the photocrosslinked hydrogel scaffolds. Whether printed or cast, a scaffold for tissue engineering comprising the hydrogel above is also subject matter of the present invention.

The resulting photocrosslinked hydrogels support the proliferation of different types of cells in vitro. Cells can advantageously also be incorporated into the bioprinting process. The cell viability, proliferation and spreading in bioprinted hydrogels are significantly better than in bulk-casted hydrogels. Thus, the photocrosslinked hydrogels are especially suited as scaffolds for tissue engineering.

The hydrogels formed from the bioink composition according to the invention, either bioprinted or casted, demonstrate good stability in vivo exceeding three months compared to animal-derived collagen which was resorbed by the natural tissues within a month.

The bioink composition according to the invention can be formulated for different 3D printing or bioprinting technologies, particularly Drop-on-Demand/jetting and digital light printing/stereolithography. The bioink is particularly suited for Drop-on-Demand printing due to its low viscosity.

The bioink is also suited towards digital light printing, where bioink compositions containing 1.5% to 6% weight of functionalized recombinant bacterial collagen-like protein according to the invention have been successfully printed into 3D hydrogel constructs.

Preferably bioink compositions containing 1.5% to 6% weight of functionalized recombinant bacterial collagen-like protein according to the invention, more preferably between 3% and 6% weight can be printed into 3D hydrogel constructs.

### EXAMPLES

### Example 1

The functionalized recombinant bacterial collagen-like protein according to the invention was prepared by reacting recombinant bacterial collagen-like protein with SEQ ID NO. 4 with methacrylic anhydride in 0.25M alkaline carbonate/hydrogencarbonate buffer at pH 9.5. Amino acids containing primary amine and hydroxyl groups can react with methacrylic anhydride to form methacrylol derivatives of methacrylamide and methacrylate respectively. The degree of functionalization is controlled via the molar ratio of recombinant bacterial collagen-like protein and methacrylic anhydride. Following the functionalization, the mixture undergoes diafiltration or dialysis to remove the side products. The degree of functionalization can be determined by trinitrobenzene sulfonic acid assay, NMR, or HPLC-MS. In the present example, trinitrobenzene sulfonic acid assay and NMR were used and the degree of functionalization was found to be between 64% and 97.3% of the primary amine groups on the recombinant bacterial collagen-like protein.

TABLE 1.1
Degree of functionalization under various molar
ratios of methacrylic anhydride to primary
amine groups of recombinant bacterial
collagen-like protein according to the invention.

| Molar ratio of methacrylic anhydride to | Degree of substitution |
|---|---|
| primary amine groups of recombinant | (based on trinitrobenzene |
| bacterial collagen-like protein | sulfonic acid assay) |
| 0.6:1 | 64.2% |
| 1:1 | 90.9% ± 2.3% |
| 2:1 | 97.2% ± 1.1% |
| 3:1 | 97.3% ± 0.6 |
| 4:1 | 93.0% ± 2.6% |

The degree of substitution is based on TNBS assay, which measures the change in primary amine groups.

### Example 2

The bioink according to the invention is formulated by dissolving the functionalized recombinant bacterial collagen-like protein from Example 1 in phosphate-buffered saline, and then adding water-soluble photoinitiator Lithium phenyl-2,4,6-trimethylbenzoylphosphinate in a concentration of 0.2% weight of the bioink formulation.

Photocrosslinkable polymers such as PEG-AR were incorporated as shown in Example 8. For optimal mixing to achieve a homogenous solution, the acellular mixture was vortexed. Due to the low viscosity and lack of pH or temperature sensitivity, it was considerably easier to obtain homogenous solution and subsequent hydrogel compared to animal-derived collagen.

Other components such as peptides, polymers, and cells were also incorporated into the formulation depending on the end application as described in Examples 6, 9 and 10.

### Example 3

The bioink composition was dispensed using a bioprinter or poured into a mould for photocuring to form hydrogel scaffolds. Photopolymerization is triggered by exposure to UV light. There is a phase transition from solution to hydrogel. The resulting hydrogels are transparent with good optical clarity and shape fidelity. Two hydrogels were produced using a bioink composition comprising 3% and 6% functionalized recombinant bacterial collagen-like protein prepared according to Example 1.

Shape fidelity refers to the shape retention of the bioink following extrusion or moulding and crosslinking (via photo or chemical methods). It compares the final structure of the hydrogel with the intended design or shape of the mould. The shape fidelity of casted hydrogels are reflected by their defined edges and smooth surfaces, particularly at higher concentrations (≥3 mg/mL).

In comparison, the shape fidelity and homogeneity of animal-derived collagen methacrylol is considerably harder to achieve as the photocrosslinking, the pH and temperature has to be well-controlled for the hydrogel to form. Non-homogenous mixing often gives rise to non-uniform hydrogels with undefined edges and bumpy surfaces.

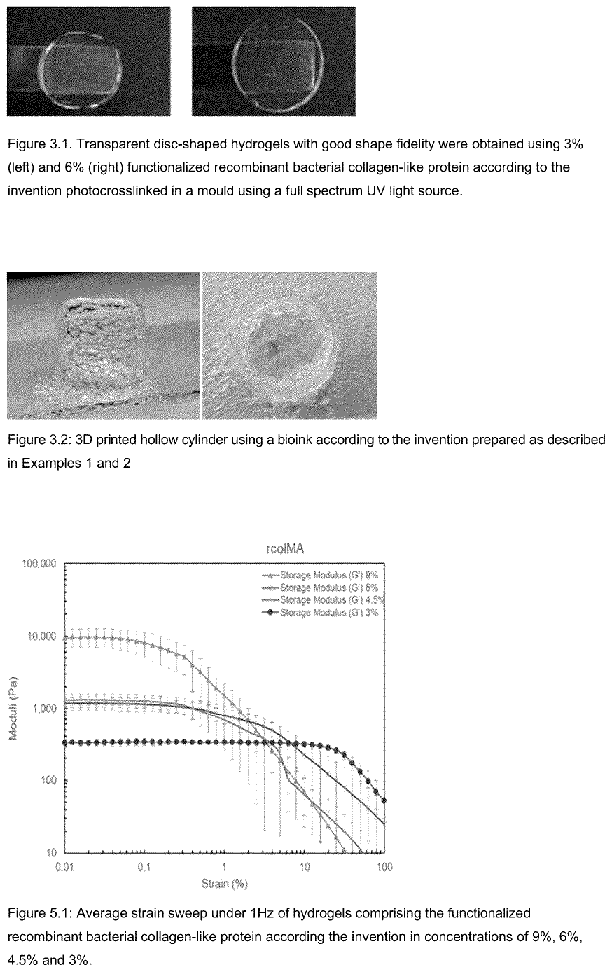

FIG. 3.1 shows transparent disc-shaped hydrogels with good shape fidelity were obtained using 3% (left) and 6% (right) functionalized recombinant bacterial collagen-like protein according to the invention photocrosslinked in a mould using a full spectrum UV light source.

The shape fidelity and printability of the material was demonstrated by 3D printing of a hollow cylinder with a height of 10.8 mm and wall thickness of 1.6 mm as shown in FIG. 3.2. Bioink containing 6% functionalized recombinant bacterial collagen-like protein according to the invention was dispensed by a Drop-on-Demand bioprinter onto a glass cover slip in a layer-by-layer format. After the deposition of each layer, the sample was photocrosslinked with UV light to induce hydrogel formation. No delamination was observed and a free-standing hollow cylinder was 3D printed.

FIG. 3.2 shows a 3D printed hollow cylinder using a bioink according to the invention prepared as described in Examples 1 and 2

### Example 4

Porous scaffolds/sponges can be prepared by freeze drying hydrogels. The hydrogels prepared using the same method as described in Example 3 with water as solvent were flash frozen in an ultra-low temperature (−80° C.) freezer. The frozen samples were then put into a lyophilizer. During the freeze-drying process, the samples were subject to low temperature and vacuum, which results in the sublimation of the water within the sample.

Sample 4.1 was prepared using a bioink composition as described in Example 2 using 0.45% weight animal collagen functionalized with methacrylol groups (comparative sample).

Sample 4.2 was prepared using a bioink composition as described in Example 2 comprising 6% weight recombinant bacterial collagen-like protein functionalized with methacryloyl groups as described in Example 1.

Sample 4.3 was prepared using a bioink composition as described in Example 2 comprising 6% weight recombinant bacterial collagen-like protein functionalized with methacryloyl groups as described in Example 1 and 5% weight of branched (8-arm) polyethylene glycol acrylate with 10 kDa molecular weight.

All samples were photocrosslinked by UV exposure.

The sponge was subsequently re-hydrated by immersing in water. Good rehydration requires overnight immersion and can be accelerated by sonication.

TABLE 4.1
Hydrogels, lyophilized sponges and re-hydrated
hydrogels prepared using bovine collagen
methacryloyl and recombinant bacterial collagen-like
protein functionalized with methacrylol groups.

| Sample | Initial hydrogel | Sponge | Re-hydrated hydrogel |
|---|---|---|---|
| 4.1 | Regular, | Disc with | Average, slightly |
| | translucent | smaller | irregular disc |
| | disc with | diameter | |
| | bumpy surface | | |
| 4.2 | Regular, | Disc with similar | Regular disc with |
| | transparent disc | dimensions | similar dimensions |
| | with smooth | as initial | as initial hydrogel |
| | surface | hydrogel | |
| 4.3 | Regular, | Shrunken disc | Regular disc |
| | transparent disc | with diameter | with similar |
| | with smooth | ⅔ of initial | dimensions as |
| | surface | hydrogel | initial hydrogel |

The sponge is a structure resulting from lyophilization. It is a porous, opaque sponge-like scaffold. The sponges have a similar shape and dimension (diameter and area) compared to the starting hydrogel. More shrinkage is observed if polymers such as PEG are incorporated in the hydrogel formulation, but the overall shape is well-maintained as the shrinkage is uniform.

### Example 5

To determine the viscoelastic properties, 15 mm disc-shaped hydrogels produced as described in Example 3 were subjected to dynamic time, strain and frequency sweep experiments using the Anton Paar MCR 302 rheometer with a 15.0 mm-diameter profiled parallel plate geometry.

Strain sweep experiments characterize hydrogels by using increasing oscillatory strain from 0.01% to 100% at a constant frequency of 1 Hz. The results of a strain sweep test are expressed via the storage (G′) and loss (G″) moduli of the hydrogel over an increasing strain range, and shows the linear viscoelastic region of the material in which the elastic behaviour (G′) predominates. The crossover point of G′ and G″ characterises the conditions which the sample has broken down and is no longer regarded as a hydrogel. The storage moduli (G′) represents the hydrogel's rigidity and the ability to store the deformation energy in an elastic manner. This is directly related to the extent of crosslinking, in which, the higher the degree of crosslinking, the higher the storage modulus. The loss moduli (G″) represents the deformation energy that is lost by internal friction during shearing of the hydrogel sample.

The frequency sweep test is another rheological method that reflects the relationship between testing frequency and the storage (G′) and loss (G″) moduli of a material. The frequency is varied from 0.1 Hz to 10 Hz while keeping the strain constant at 0.1%. The results reflect the viscoelastic properties and state of the hydrogels by comparing the two G′ and G″ values over the frequency range.

The samples were prepared using a bioink composition as described in Example 2 comprising 9%, 6%, 4.5% and 3% weight recombinant bacterial collagen-like protein functionalized with methacrylol groups as described in Example 1.

All samples were photocrosslinked by UV exposure.

FIG. 5.1 shows an average strain sweep under 1 Hz of hydrogels comprising the functionalized recombinant bacterial collagen-like protein according the invention in concentrations of 9%, 6%, 4.5% and 3%.

FIG. 5.1 shows that the hydrogel rigidity can be tuned by varying the concentration of the functionalized recombinant bacterial collagen-like protein according to the invention. At a concentration of 3% weight recombinant bacterial collagen-like protein functionalized with methacrylol groups the rigidity (G′) was found to be 340 Pa and at a concentration of 9% it G′ was 8200 Pa.

The rigidity (G′) of the hydrogels increases with increasing concentration of recombinant bacterial collagen-like protein functionalized with methacrylol groups as described in Example 1. However, this increase is not linear. Interestingly, as the concentration of recombinant bacterial collagen-like protein functionalized with methacrylol groups increases, the strain at which the crossover point occurs decreases. This demonstrates that the higher concentration hydrogels are more brittle. The 3% hydrogel can withstand higher strain before the structure breaks down compared with the 4.5%, 6% and 9% hydrogels.

Due to solubility limitations and pH-induced gelation, hydrogels produced from animal-derived collagen methacrylol are typically less than 0.6% w/v. Consequently, the mechanical properties are significantly weaker, with G′ values of 118 Pa.

### Example 6

Example 6 shows that the hydrogels comprising functionalized recombinant bacterial collagen-like protein according to the invention are cytocompatible. The experiments were conducted with human dermal fibroblasts under standard cell culture conditions. The hydrogels were produced as described in Examples 1 to 3.

The cytocompatibility of recombinant bacterial collagen-like protein functionalized with methacrylol groups as described in Example 1 was evaluated and compared against controls which were the standard culture (no protein) and non-functionalized recombinant bacterial collagen-like protein. The test samples were first dissolved in cell culture media and subsequently diluted to different concentrations. Human dermal fibroblasts cultured in 96-well plates were exposed to different dilutions of the test samples for 24 hours, under standard culture conditions. The cellular metabolic activity, an indicator of cell viability, proliferation and cytotoxicity of the test sample, was quantified. The colorimetric XTT assay was implemented as the metabolically active cells would reduce the yellow tetrazolium salt to an orange formazan dye. The concentration of the orange formazan dye was measured via absorbance measurements at 450 nm by a microplate reader.

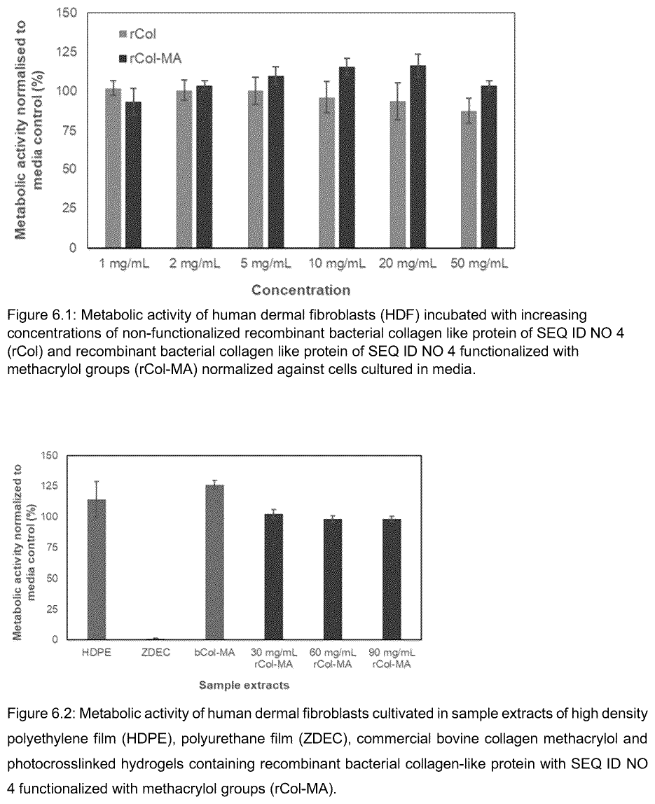

FIG. 6.1 shows the metabolic activity of human dermal fibroblasts (HDF) incubated with increasing concentrations of non-functionalized recombinant bacterial collagen like protein of SEQ ID NO 4 (rCol) and recombinant bacterial collagen like protein of SEQ ID NO 4 functionalized with methacrylol groups (rCol-MA) normalized against cells cultured in media.

Solutions of non-functionalized recombinant bacterial collagen-like protein of SEQ ID NO: 4 (rCol) and recombinant bacterial collagen-like protein of SEQ ID NO: 4 functionalized with methacrylol groups (rCol-MA) are cytocompatible, as evaluated by incubating human dermal fibroblasts (HDF) with increasing concentrations of sample dissolved in media for 24 hours under standard cell culture conditions. The metabolic activity was quantified using XTT assay and then normalized against cells cultured in media.

The metabolic activity of cells exposed to test solutions at all the tested concentrations were comparable to cells cultured under normal conditions. This indicates that recombinant bacterial collagen-like protein and recombinant bacterial collagen-like protein functionalized with methacrylol groups are cytocompatible and do not induce cell toxicity.

The cytocompatibility of hydrogels prepared from photocrosslinked recombinant bacterial collagen-like protein functionalized with methacrylol groups, prepared as described in Example 2, were also evaluated. High density polyethylene film (HDPE) and a commercial bovine collagen methacrylate (bCol-MA) were used as negative controls while polyurethane film (ZDEC) was used as a positive control to evaluate cytotoxicity. Extracts of the samples were prepared by incubating the samples in cell culture media for 72 hours at 37° C. The protocol was based on the international standard DIN EN ISO 10993-12. The extracts were then administered to human dermal fibroblasts for 24 hours, following which the metabolic activity was measured using XTT assay as described above.

FIG. 6.2 shows the metabolic activity of human dermal fibroblasts cultivated in sample extracts of high density polyethylene film (HDPE), polyurethane film (ZDEC), commercial bovine collagen methacrylol and photocrosslinked hydrogels containing recombinant bacterial collagen-like protein with SEQ ID NO 4 functionalized with methacrylol groups (rCol-MA).

Hydrogels prepared from photocrosslinked recombinant bacterial collagen-like protein functionalized with methacrylol groups are cytocompatible, regardless of concentration. The metabolic activity of cells exposed to extracts of these hydrogels were comparable to that of cells cultured under normal conditions and also cells cultured in extracts of HDPE. Extracts prepared from hydrogels of bovine collagen functionalized with methacryloyl induced greater metabolic activity and cell proliferation. In contrast, the positive control ZDEC induced significant cell toxicity as the cellular metabolic activity dropped to baseline. The results of the XTT were corroborated with fluorescent microscopy observations of the live cells.

Cells were also cultured on and encapsulated within hydrogels prepared from photocrosslinked recombinant bacterial collagen-like protein functionalized with methacrylol groups according to the invention. The hydrogels were prepared as described in Example 3. When human dermal fibroblasts were cultured on hydrogels, increasing the concentration of recombinant bacterial collagen-like protein functionalized with methacrylol groups from 3% to 6% promoted faster proliferation and greater spreading of cells. This was observed via fluorescent imaging of cells seeded onto the hydrogels. In contrast, when human dermal fibroblasts were encapsulated within the hydrogels, faster cell proliferation and increased cell spreading was observed for cells encapsulated in lower concentration hydrogels of 1.5% compared to cells cultured in 3% and 6% hydrogels. This was attributed to the lower rigidity of 1.5% hydrogels which promoted cell migration and spreading.

### Example 7

Example 7 was carried out to investigate the biocompatibility of the hydrogels and sponges comprising functionalized recombinant bacterial collagen-like protein according to the invention in vivo. The hydrogels and sponges were produced as described in Examples 1 to 3.

Hydrogels and sponges prepared from functionalized recombinant bacterial collagen-like protein according to the invention were implanted into the subcutaneous space of immunocompetent C57BL/6 mice. Controls include biocompatible high density polyethylene film (HDPE) and sham surgery without implant.

The implants and surrounding tissue were explanted after 1- and 3-months to ascertain the biocompatibility, stability and irritation potential of the implant material. Histological examinations of the explants were performed according to ISO 10993-6:2009.

At the 1-month timepoint, all the samples can still be observed in the explanted tissue. Varying degrees of cell infiltration by polymorphonuclear cells, lymphocytes, plasma cells, macrophages and some giant cells were observed. Newly formed blood vessels were also observed.

At the 3-month timepoint, all the photocrosslinked scaffolds and sponges were still observed in the explant, suggesting in vivo stability.

Comparing the immune response of the hydrogel and sponge samples with the HDPE and sham controls, the photocrosslinked recombinant bacterial collagen-like protein functionalized with methacrylol groups is considered a non-irritant.

### Example 8

The recombinant bacterial collagen like protein of SEQ ID NO 4 functionalized with methacrylol groups (rCol-MA) was mixed with polyethylene glycol acrylates (PEG-AR), and subsequently photocrosslinked to form hydrogels. Such formulations allow the reduction in the amount of functionalized recombinant bacterial collagen-like protein according to the invention needed for hydrogel formation, as well as enable the modulation of mechanical and physical properties.

Different hydrogels were produced using varying ratios of the recombinant bacterial collagen-like protein functionalized with methacryloyl groups as shown in Example 1 and branched (8-arm) PEG-AR of molecular weight 10 kDa.

Sample 8.1 was prepared using a bioink composition as described in Example 2 comprising 6% recombinant bacterial collagen like protein of SEQ ID NO 4 functionalized with methacrylol groups.

Sample 8.2 was prepared using a bioink composition comprising 6% branched polyethylene glycol acrylate of 10 kDa molecular weight.

Sample 8.3 was prepared using a bioink composition as described in Example 2 comprising 3% recombinant bacterial collagen like protein of SEQ ID NO 4 functionalized with methacrylol groups and 3% branched polyethylene glycol acrylate of 10 kDa molecular weight.

Sample 8.4 was prepared using a bioink composition as described in Example 2 comprising 6% recombinant bacterial collagen like protein of SEQ ID NO 4 functionalized with methacrylol groups and 3% branched polyethylene glycol acrylate of 10 kDa molecular weight.

Sample 8.5 was prepared using a bioink composition as described in Example 2 comprising 6% recombinant bacterial collagen like protein of SEQ ID NO 4 functionalized with methacrylol groups and 6% branched polyethylene glycol acrylate of 10 kDa molecular weight.

TABLE 8.1
Averaged storage, loss moduli and corresponding standard deviation
at 0.1% strain of hydrogels formulated with different ratios of
functionalized recombinant bacterial collagen-like protein
according to the invention and branched polyethylene glycol
acrylate of 10 kDa molecular weight. The values provided
are the average of triplicate measurements

| | Storage modulus | Loss modulus at |
|---|---|---|
| Hydrogel | at 0.1% | 0.1% strain, |
| sample | strain, G′_{0.1} (Pa) | G′′_{0.1} (Pa) |
| 8.1 | 1154.5 (±247) | 521 (±12) |
| 8.2 | 1820 (±1607) | 79.8 (±60) |
| 8.3 | 1710 (±42) | 47.4 (±12) |
| 8.4 | 5930 (±3945) | 385 (±101) |
| 8.5 | 33200 (±6693) | 1191 (±329) |

At the same concentration of 6%, the branched polyethylene glycol acrylate hydrogel and functionalized recombinant bacterial collagen-like protein according to the invention have comparable rigidities. When 3% functionalized recombinant bacterial collagen-like protein according to the invention is formulated with 3% branched polyethylene glycol acrylate, the resulting hydrogel has similar G′. This shows that addition of branched polyethylene glycol acrylate compensated for a reduction in concentration of functionalized recombinant bacterial collagen-like protein according to the invention, while maintaining the mechanical properties.

Keeping the concentration of functionalized recombinant bacterial collagen-like protein according to the invention consistent at 6%, increasing the concentration of branched polyethylene glycol acrylate increases the rigidity of the resulting hydrogel.

### Example 9

The recombinant bacterial collagen-like protein of SEQ ID NO 4 functionalized with methacrylol groups was mixed with suspensions of biosynthetic nanocellulose fibers (NF) and subsequently photocrosslinked to form composite hydrogels.

The NF is produced by bacterial fermentation. Stock solutions of NF was mixed with stock solutions of recombinant bacterial collagen-like protein functionalized with methacryloyl groups as shown in Example 1 (rCol-MA) and with photoinitiator. NF was incorporated into this composite mixture up to 0.6% weight together with 1.5% recombinant bacterial collagen like protein of SEQ ID NO 4 functionalized with methacrylol groups and 0.2% Lithium phenyl-2,4,6-trimethylbenzoylphosphinate (LAP) photoinitiator. The addition of NF increased the viscosity of the mixture, as determined by rheology measurements using an Anton Paar MCR 302 rheometer with a 15 mm profiled measuring plate. Shear thinning property was also observed for NF/rCol-MA mixtures. All these viscosity characteristics makes the formulation highly suited for potential applications such as injectable hydrogels and bioinks for extrusion-based bioprinting.

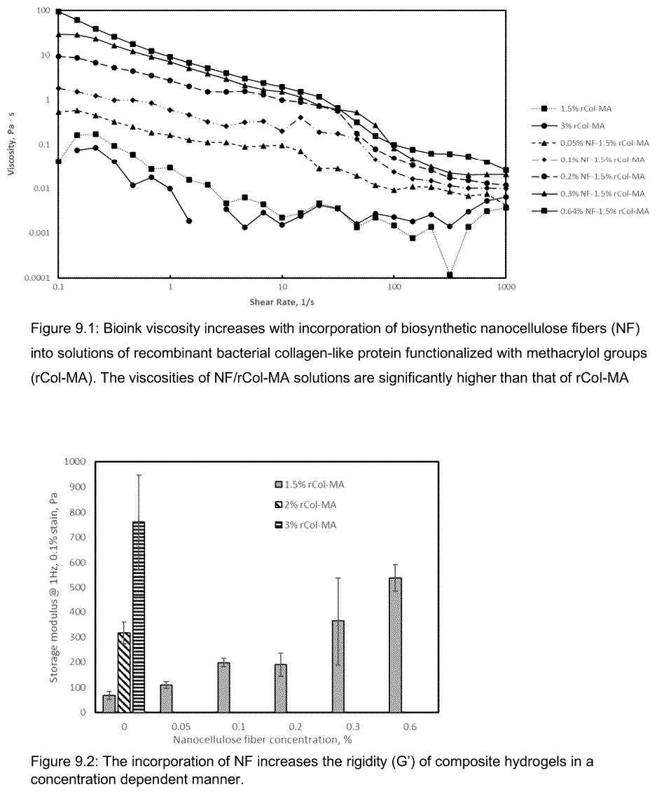

FIG. 9.1 illustrates how bioink viscosity increases with incorporation of biosynthetic nanocellulose fibers (NF) into solutions of recombinant bacterial collagen-like protein functionalized with methacrylol groups (rCol-MA). The viscosities of NF/rCol-MA solutions are significantly higher than that of rCol-MA solutions. For all solution mixtures, the viscosity increases with the increase in NF content. Shear-thinning response, i.e. decreasing of viscosity with the increase in shear rate, was observed in all NF/rCol-MA solutions.

Following photocrosslinking, the nanocellulose fibers mechanically reinforce the soft hydrogels, particularly at low concentrations of 1.5% of the recombinant bacterial collagen-like protein functionalized with methacryloyl groups as shown in Example 1 (rCol-MA). The rigidity of different formulations were compared in strain sweep studies, at 0.1% strain (within the linear viscoelastic region) and 1 Hz frequency.

FIG. 9.2 depicts how the incorporation of NF increases the rigidity (G′) of composite hydrogels in a concentration dependent manner.

The storage modulus of the hydrogel increases with the concentration of functionalized recombinant bacterial collagen-like protein according to the invention. For 1.5% rCol-MA hydrogel, the average storage modulus is around 70 Pa, while for 3%, the value increases to around 760 Pa. Incorporation of NF also increases the storage modulus of rCol-MA containing hydrogels. The storage modulus of composite hydrogel with 0.3% NF and 1.5% rCol-MA is similar to that of 2% rCol-MA hydrogel.

The reinforced composite hydrogels offer a favorable microenvironment for the proliferation, spreading and migration of encapsulated human dermal fibroblast cells.

### Example 10

The functionalized recombinant bacterial collagen-like protein prepared as shown in Example 1 was blended with short self-assembling peptide Ac-KGAVLI-NH₂ to form a composite hydrogel which is chemically well-defined. Such composite systems combine the advantages of both the photocrosslinkable functionalized recombinant bacterial collagen-like protein according to the invention and the self-assembling peptides. The propensity of the peptides to self-assemble under defined conditions (addition of a saline buffer or increase in temperature) allow for hydrogel formation, even when the concentration of the functionalized recombinant bacterial collagen-like protein according to the invention is below the gelation limit or in the absence of photocrosslinking.

Surprisingly, the functionalized recombinant bacterial collagen-like protein according to the invention (despite its significantly larger molecular mass) did not interfere with the self-assembly of the peptide as the minimum gelation concentration of 5 mg/mL (0.5% weight) peptide were comparable in the presence and absence of functionalized recombinant bacterial collagen-like protein according to the invention. This is an unexpected result as peptide self-assembly is driven by intermolecular interactions which are disrupted by the addition of bulky proteins such as serum albumin. Moreover, significantly higher concentrations of functionalized recombinant bacterial collagen-like protein according to the invention could be encapsulated compared to the concentration of the peptide, even at the minimum gelation concentrations of the peptide. Post-gelation UV can be applied to further “cure” the hydrogel. The photocrosslinked composite hydrogel has improved mechanical properties compared to hydrogels containing only the peptide and hydrogels containing only photocrosslinked functionalized recombinant bacterial collagen-like protein prepared as shown in Example 1.

The hexameric N-terminal acetylated, C-terminal amidated peptide Lysine-Glycine-Alanine-Valine-Leucine-Isoleucine (Ac-KGAVLI-NH2) was selected as the optimal peptide component due to its propensity to consistently form hydrogels under physiological conditions at concentrations as low as 5 mg/mL (0.5%). This peptide can encapsulate significantly larger amounts of functionalized recombinant bacterial collagen-like protein according to the invention.

### Hydrogels

containing 0.5% peptide Ac-KGAVLI-NH2,
containing 0.5% peptide Ac-KGAVLI-NH2 and 3% functionalized recombinant bacterial collagen-like protein according to the invention and
containing 0.5% peptide Ac-KGAVLI-NH2 and 3% functionalized recombinant bacterial collagen-like protein according to the invention that was UV cured
   were visually evaluated. The incorporation of functionalized recombinant bacterial collagen-like protein according to the invention gave rise to less opaque hydrogels which gelled more rapidly.

In addition, the composite hydrogels have higher rigidity compared to hydrogels containing only peptide and hydrogels containing photocrosslinked functionalized recombinant bacterial collagen-like protein prepared as shown in Example 1.

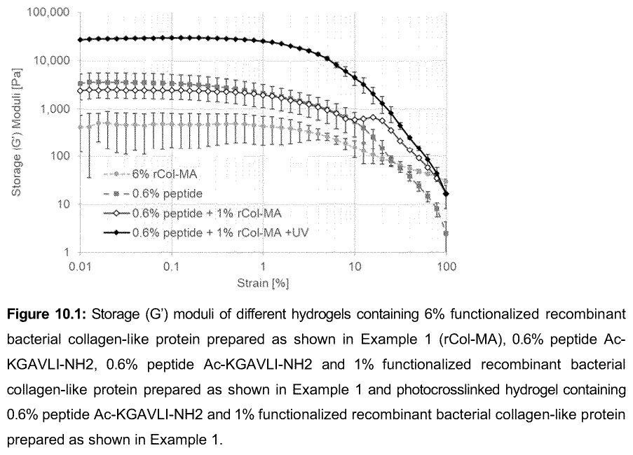

FIG. 10.1 shows storage (G′) moduli of different hydrogels containing 6% functionalized recombinant bacterial collagen-like protein prepared as shown in Example 1 (rCol-MA), 0.6% peptide Ac-KGAVLI-NH2, 0.6% peptide Ac-KGAVLI-NH2 and 1% functionalized recombinant bacterial collagen-like protein prepared as shown in Example 1 and photocrosslinked hydrogel containing 0.6% peptide Ac-KGAVLI-NH2 and 1% functionalized recombinant bacterial collagen-like protein prepared as shown in Example 1.

The hydrogels containing 0.6% peptide Ac-KGAVLI-NH2 had significantly higher storage moduli values of 3 kPa compared to hydrogels containing 6% weight photocrosslinked functionalized recombinant bacterial collagen-like protein prepared as shown in Example 1 (0.5 kPa). A composite hydrogel containing 0.6% peptide Ac-KGAVLI-NH2 and 1% functionalized recombinant bacterial collagen-like protein prepared as shown in Example 1 increased the rigidity by 3 times. The rigidity of the composite hydrogels containing functionalized recombinant bacterial collagen-like protein prepared as shown in Example 1 can be further enhanced via photocrosslinking following exposure to UV in the presence of a photoinitiator.

Composite hydrogels containing hexameric peptide Ac-KGAVLI-NH2 and functionalized recombinant bacterial collagen-like protein according to the invention are also biocompatible. Human dermal fibroblasts were uniformly encapsulated within a 0.6% peptide Ac-KGAVLI-NH2+1% functionalized recombinant bacterial collagen-like protein prepared as shown in Example 1 composite hydrogel. The cells proliferated and spread normally and evidently showed by their spindle-like morphology. Thus, the composite hydrogels are suitable for long term culture.

### PROTEIN SEQUENCES

SEQ ID NO:1 Streptococcus pyogenes Collagen-like protein (CLP), full length protein
SEQ ID NO:2 Streptococcus pyogenes CLP, truncation 3
SEQ ID NO:3 Streptococcus pyogenes CLP, truncation 5
SEQ ID NO:4 Streptococcus pyogenes CLP, no V-domain

1. Recombinant bacterial collagen-like protein with an amino acid sequence that is at least ≥60% identical to the amino acid sequence of SEQ ID NO:1 characterized in that the amino acid sequence comprises a deletion of at least 38 amino acids at the N-terminus of the amino acid sequence of SEQ ID NO:1 and that the recombinant collagen-like protein is functionalized with ethylenic unsaturated groups.

2. Recombinant bacterial collagen-like protein functionalized with ethylenic unsaturated groups according to claim 1, characterized in that the amino acid sequence is at least 60% identical to the amino acid sequence of SEQ ID NO:2, SEQ ID NO:3 or SEQ ID NO:4.

3. Recombinant bacterial collagen-like protein functionalized with ethylenic unsaturated groups according to claim 2, characterized in that the amino acid sequence is at least 90% identical to the amino acid sequence of SEQ ID NO:2, SEQ ID NO:3 or SEQ ID NO:4.

4. Recombinant bacterial collagen-like protein functionalized with ethylenic unsaturated groups according to claim 1, characterized in that the degree of functionalization ranges from 5% to 99% of the primary amine groups on the N-terminus, the primary amine groups of the lysine residues and the hydroxyl groups of the tyrosine residues of the recombinant collagen-like protein.

5. Recombinant bacterial collagen-like protein functionalized with ethylenic unsaturated groups according to claim 2, characterized in that the ethylenic unsaturated groups are (meth)acrylol, fumarate or vinyl groups.

6. Recombinant bacterial collagen-like protein functionalized with ethylenic unsaturated groups according to claim 5, characterized in that the ethylenic unsaturated groups are methacrylate, acrylate, methacrylamide, acrylamide or mixtures thereof.

7. Recombinant bacterial collagen-like protein functionalized with ethylenic unsaturated groups according to claim 6, characterized in that the amino acid sequence at least 98% identical to the amino acid sequence of SEQ ID NO:2, SEQ ID NO:3 or SEQ ID NO:4 and the recombinant collagen-like protein is functionalized with methacrylate, acrylate, methacrylamide, acrylamide groups or mixtures thereof.

8. Recombinant bacterial collagen-like protein functionalized with ethylenic unsaturated groups according to claim 7, characterized in that the amino acid sequence at least 99% identical to the amino acid sequence of SEQ ID NO:2, SEQ ID NO:3 or SEQ ID NO:4 and the recombinant collagen-like protein is functionalized with methacrylate, acrylate, methacrylamide, acrylamide groups or mixtures thereof.

9. Process for functionalizing a recombinant bacterial collagen-like protein with an amino acid sequence at least 98% identical to the amino acid sequence of SEQ ID NO:2, SEQ ID NO:3 or SEQ ID NO:4 with (meth)acrylic anhydride, characterized in that functionalization is carried out at a molar ratio of (meth)acrylic anhydride to amine groups of the recombinant bacterial collagen-like protein between 0.5:1 and 5:1.

10. Bioink formulation comprising
a) 1 to 10% weight of functionalized recombinant bacterial collagen-like protein according to claim 1
b) 80 to 99% weight of aqueous solvent
c) 0.01 to 2% weight of a photoinitiator
d) 0 to 5% weight of photocrosslinkable polymers or other photocrosslinkable peptides
e) 0 to 10% weight of additives, rheology modifiers, biopolymers, gelation enhancers, bioactive moieties, peptides, nanocellulose and/or cells
provided that the sum of all components of the bioink are 100%.

11. Process for producing a hydrogel by photocrosslinking the functionalized recombinant bacterial collagen-like protein according to claim 1.

12. Process for producing a hydrogel by photocrosslinking the bioink formulation according to claim 10.

13. Process for producing a hydrogel according to claim 12, characterized in that the bioink formulation is photocrosslinked using UV light.

14. Hydrogel comprising a photocrosslinked functionalized recombinant bacterial collagen-like protein according to claim 1.

15. Hydrogel according to claim 14 characterized in that the hydrogel further comprises nanocellulose, peptides or mixtures thereof.

16. A scaffold for tissue engineering comprising the hydrogel according to claim 11.


SEQUENCE LISTING (5 sequences)

SEQ ID NO: 1 - protein, 330 aa, Streptococcus pyogenes
NHKVHMHHHHHHADEQEEKAKVRTELIQELAQGLGGIEKKNFPTLGDEDLDHTYMTKLLTYLQEREQAENSWRKRLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 2 - protein, 292 aa, Streptococcus pyogenes
NHKVHMHHHHHHDHTYMTKLLTYLQEREQAENSWRKRLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 3 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 4 - protein, 240 aa, Streptococcus pyogenes
GSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 5 - protein, 6 aa, synthetic construct
KGAVLI